(19) United States
(12) Plant Patent Application Publication
(10) Pub. No.: US 2010/0024086 P1
(43) Pub. Date: Jan. 28, 2010

(54) Poa pratensis L. named 'NE-KYB-05-001'

(50) ;

(76) Inventors: Robert C. Shearman, Lincoln, NE (US); Terrance P. Riordan, Lincoln, NE (US); Leonard A. Wit, JR., Bennet, NE (US)

Correspondence Address: Sod Solutions, Inc., P.O. Box 460, Mount Pleasant, SC, 29465, US

(21) Appl. No.: 12/460,378

(22) Filed: Jul. 17, 2009

Related U.S. Application Data
(60) Provisional application No. 61/135,114, filed on Jul. 17, 2008.

(51) Int. Cl. A01H 5/00

(52) U.S. Cl. PLT/393

(57) ABSTRACT

A new and distinct variety of Kentucky Bluegrass named ‘NE-KYB-05-001’ (trade name Bella), is characterized by its vegetative only propagation, improved shade and drought tolerance, dwarf-like and dense growth habit, dark green foliage and shorter leaves compared to other Kentucky bluegrass varieties.

### LATIN NAME OF THE GENUS AND SPECIES

The Latin name of the genus and species of the novel variety disclosed herein is Poa pratensis L.

### VARIETY DENOMINATION

The inventive variety of Poa pratensis L. disclosed herein has been given the variety denomination ‘NE-KYB-05-001’.

### BACKGROUND OF THE INVENTION

The present invention relates to a new and distinct perennial variety of Poa pratensis L.

‘NE-KYB-05-001’ is a result from the breeding program at the University of Nebraska, Lincoln. The parent grasses are unknown at the current time. ‘NE-KYB-05-001’ has been vegetatively propagated since it is a poor seed producer, which makes it unique and different than any other Poa pratensis L. It is anticipated that the plant of this invention will be marketed under the synonym Bella as a trade name. ‘NE-KYB-05-001’ is so identified in pictures and morphological and agronomic charts of this disclosure.

### SUMMARY OF THE INVENTION

‘NE-KYB-05-001’ is a distinctive variety of Poa pratensis L. being a dark green, extremely low-growing Kentucky bluegrass cultivar, having superior high temperature adaptation and drought resistance. ‘NE-KYB-05-001’ is a very poor seed producer, making it suitable for vegetative reproduction only, such as sprigs, rhizomes, plantlets, or sod. ‘NE-KYB-05-001’ is adapted for use in the cool-humid, cool-arid areas in the US and Canada, zones 2 to 7 of the Plant Heat Zone Map, and transition zone 8 (FIG. 1).

### BRIEF DESCRIPTION OF THE FIGURES

FIG. 1: ‘NE-KYB-05-001’ in a shade study at UN; Mead, Nebr.

FIG. 2: ‘NE-KYB-05-001’ field at Newton, Ga. (transition zone 8).

FIG. 3: ‘NE-KYB-05-001’ test plots at MSU, Miss. ‘NE-KYB-05-001’ is first on the left at lower corner, in the center of the plots and at right upper corner.

FIG. 4: ‘NE-KYB-05-001’ test plots at UN, Nebr. ‘NE-KYB-05-001’ is on 1st row second to left, 2nd row 3rd from left and 3rd row 1st from left.

FIG. 5: ‘NE-KYB-05-001’ close up.

FIG. 6: ‘NE-KYB-05-001’ plug.

FIG. 7: Plant height comparison: ‘NE-KYB-05-001’ top; Midnight bottom.

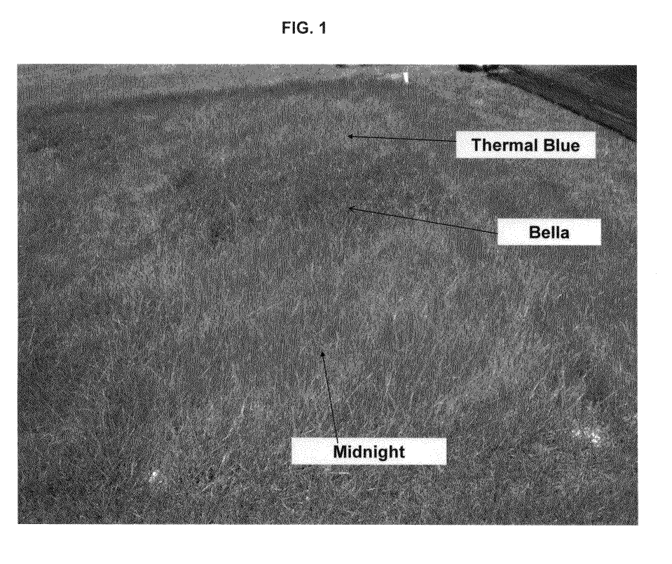

### DESCRIPTION OF THE NEW VARIETY

The following is a detailed description of the new grass variety, based upon observations of the plant grown in field plots at the University of Nebraska, John Seaton Anderson Turfgrass Research Facility located near Mead, Nebr. ‘NE-KYB-05-001’ is an outstanding Kentucky bluegrass cultivar that is drought resistant and tolerant of high temperatures. Its drought resistance characteristics make it suited for use where water conservation is an issue. Its heat tolerance makes it suitable for use in the transition zone. ‘NE-KYB-05-001’ is low-growing and requires infrequent mowing, making it an excellent choice for lawns which can benefit from reduced mowing schedule and for sites that are difficult to mow, such as slopes and banks. ‘NE-KYB-05-001’ requires low fertilizer input and grows well on soils ranging from sand to heavy clay. ‘NE-KYB-005-001’ can only be established vegetatively by sprigs, rhizomes, plugs or sod. ‘NE-KYB-05-001’ has also an excellent shade tolerance compared to other bluegrasses. Its dark green color and low growth habit give it a highly appealing turfgrass quality and appearance.

### Growth Habit and Dimensions

‘NE-KYB-05-001’ was compared to 19 other cultivars of Kentucky bluegrass at the University of Nebraska John Seaton Anderson Turfgrass Research Facility located near Mead, Nebr. starting in September 2005. Treatments for all cultivars were the same.

The Analysis of Variance (ANOVA) indicated that cultivars differed significantly for most variables measured (Table 1). Cultivars were quite similar in days to flowering, but ‘NE-KYB-05-001’ flowered later than Blue Moon, Absolute, and Kenblue (Table 2). ‘NE-KYB-05-001’ is a low growing cultivar. It had the lowest foliage height of any of the cultivars studied (Table 2). ‘NE-KYB-05-001’ had nearly half the plant foliage height of the nearest low growing cultivar. In addition to the low foliage height, ‘NE-KYB-05-001’ had the shortest leaf length of any of the cultivars (Table 2).

‘NE-KYB-05-001’ has a dwarf-like growth habit, which would require less frequent mowing. ‘NE-KYB-05-001’ ranked intermediate in leaf width with Princeton 105 having greater leaf width and Park having finer leaf width. Even though ‘NE-KYB-05-001’ has a dwarf-like growth habit, it still ranked among the cultivars with the greatest amount of plant spread (Table 2). ‘NE-KYB-05-001’, Absolute, and NuDensity had the lowest incidence of stem rust [Puccinia graminis f.sp. poae (Pers.)], which was the only disease that occurred during this study.

‘NE-KYB-05-001’ had the lowest seed yield (110 lbs acre⁻¹) of the cultivars studied, and it had the poorest seed germination (12%) (Table 3). Both of these characteristics make it impractical to produce ‘NE-KYB-05-001’ as a seeded cultivar; therefore ‘NE-KYB-05-001’ has to be reproduced vegetatively. ‘NE-KYB-05-001’ and Absolute had the shortest panicle length, and ‘NE-KYB-05-001’ had the lowest flag leaf height, and shortest flag leaf length of the cultivars studied (Table 3).

TABLE 1
Analysis of Variance for Kentucky bluegrass cultivar comparisons
with ‘NE-KYB-05-001’ conducted during the 2005 to 2007 growing seasons.

| | | Mean Squares of the traits | | | | | | | | | | |
|---|---|---|---|---|---|---|---|---|---|---|---|---|
| Source | df | HD^{†} | FD | FH | LL | LW | PL | PS | FLH | FLL | FLW | SR |
| Rep | 2 | 6.07 | 45.2 | 55.6 | 12.1 | 0.0006 | 4.48 | 20.42 | 3.88 | 15.93 | 0.013 | 3.03 |
| Variety | 19 | 90.5 | 24.7 | 141.2^{**} | 34.9^{**} | 0.002^{**} | 6.79^{**} | 6.28^{**} | 48.71^{**} | 8.65^{**} | 0.007^{*} | 1.44^{**} |
| Error | 38 | 17.9 | 7.3 | 15.1 | 2.6 | 0.0003 | 0.9 | 2.8 | 7.5 | 3.0 | 0.003 | 0.3 |
| CV | | 4.1 | 2.4 | 12.4 | 9.1 | 3.9 | 9.9 | 11.4 | 17.7 | 25.2 | 9.7 | 18.2 |

^{†}HD—Heading Date
FD—Flowering Date
FH—Foliage Height
LL—Leaf Length
LW—Leaf Width
PL—Panicle Length
PS—Plant Spread
FLH—Flag Leaf Height
FLL—Flag Leaf Length
FLW—Flag Leaf Width
SR—Stem Rust
^{*}Significant at 5% level
^{**}Significant at 1% level

TABLE 2
Kentucky bluegrass cultivar comparisons with ‘NE-KYB-05-001’
conducted during the 2005 to 2007 growing season.

| | | | | | | | | Stem |
|---|---|---|---|---|---|---|---|---|
| | | HD | FD | FH | LL | LW | PS | Rust |
| No | Variety | (days) | (days) | (cm) | (cm) | (cm) | (cm) | (1-9) |
| 1 | Absolute | 99 | 108 | 30.3 | 16.80 | 0.47 | 14.8 | 1.9 |
| 2 | ‘NE-KYB- | 108 | 115 | 13.3 | 9.20 | 0.47 | 15.4 | 1.9 |
| | 05-001’ | | | | | | | |
| 3 | Blue Moon | 98 | 109 | 31.7 | 17.20 | 0.45 | 14.9 | 3.9 |
| 4 | Caliber | 97 | 115 | 48.9 | 25.80 | 0.48 | 16.9 | 3.0 |
| 5 | Common | 98 | 114 | 37.3 | 19.60 | 0.45 | 15.1 | 3.6 |
| | 98/80 | | | | | | | |
| 6 | Everest | 109 | 116 | 25.1 | 14.10 | 0.45 | 13.2 | 4.3 |
| 7 | Everglade | 111 | 116 | 29.5 | 16.50 | 0.44 | 13.6 | 2.6 |
| 8 | Impact | 106 | 113 | 28.7 | 16.30 | 0.44 | 11.8 | 2.5 |
| 9 | Kenblue | 92 | 106 | 39.3 | 22.40 | 0.44 | 17.1 | 3.6 |
| 10 | Liberator | 105 | 112 | 26.5 | 15.50 | 0.45 | 14 | 3.3 |
| 11 | Midnight | 93 | 113 | 31.8 | 20.80 | 0.43 | 17.1 | 3.0 |
| 12 | NuBlue | 102 | 116 | 35.4 | 20.10 | 0.44 | 15.4 | 3.9 |
| 13 | NuDensity | 104 | 113 | 30.8 | 16.00 | 0.43 | 13.7 | 1.9 |
| 14 | NuGlade | 107 | 115 | 27.6 | 16.30 | 0.45 | 12.4 | 3.3 |
| 15 | Park | 97 | 113 | 36.4 | 19.80 | 0.42 | 15.1 | 3.2 |
| 16 | Perfection | 111 | 119 | 29.8 | 16.90 | 0.44 | 15.4 | 2.5 |
| 17 | Princeton | 103 | 113 | 34.2 | 20.30 | 0.52 | 14.6 | 2.4 |
| | P-105 | | | | | | | |
| 18 | Rugby II | 100 | 112 | 34.9 | 17.00 | 0.45 | 14.6 | 3.4 |
| 19 | Total Eclipse | 105 | 112 | 27.9 | 15.90 | 0.43 | 13.4 | 3.3 |
| 20 | Tsunami | 104 | 113 | 30.8 | 16.90 | 0.43 | 13.6 | 3.3 |
| | LSD (5%) | 6.9 | 4.5 | 6.4 | 2.7 | 0.03 | 2.8 | 0.9 |

^{†}HD—Heading Date (days to heading),
FD—Flowering Date (days to flowering),
FH—Foliage Height (cm),
LL—Leaf Length (cm),
LW—Leaf Width (cm),
PS—Plant Spread (cm), and
SR—Stem Rust (1-9 visual rating with 1 = 0-10% and 9 = 90-100% disease incidence)

TABLE 3
Kentucky bluegrass cultivar comparisons with ‘NE-KYB-05-001’
conducted during the 2005 to 2007 growing seasons.

| | | | | | | SY | |
|---|---|---|---|---|---|---|---|
| | | PL | FLH | FLL | FLW | (lbs | PB |
| No | Variety | (cm) | (cm) | (cm) | (cm) | acre⁻¹) | (%) |
| 1 | Absolute | 7.6 | 14.0 | 5.1 | 0.59 | 650 | 92 |
| 2 | ‘NE-KYB-05-001’ | 7.6 | 10.2 | 3.5 | 0.59 | 110 | 12 |
| 3 | BlueMoon | 9.7 | 12.6 | 6.2 | 0.53 | 810 | 92 |
| 4 | Caliber | 14.9 | 24.7 | 9.3 | 0.54 | 711 | 92 |
| 5 | Common98/80 | 9.9 | 20.7 | 8.0 | 0.49 | 749 | 85 |
| 6 | Everest | 8.4 | 11.2 | 5.7 | 0.59 | 668 | 92 |
| 7 | Everglade | 8.7 | 11.8 | 6.8 | 0.59 | 679 | 88 |
| 8 | Impact | 9.2 | 19.2 | 8.1 | 0.55 | 839 | 80 |
| 9 | Kenblue | 9.9 | 21.7 | 7.3 | 0.45 | 767 | 85 |
| 10 | Liberator | 8.6 | 13.0 | 6.0 | 0.53 | 709 | 90 |
| 11 | Midnight | 9.9 | 16.9 | 7.0 | 0.47 | 279 | 90 |
| 12 | MN Park | 9.6 | 19.3 | 7.6 | 0.49 | 630 | 88 |
| 13 | NuBlue | 9.0 | 15.5 | 5.3 | 0.49 | 813 | 90 |
| 14 | NuDensity | 8.6 | 12.2 | 7.2 | 0.58 | 875 | 88 |
| 15 | NuGlade | 9.2 | 13.6 | 6.4 | 0.61 | 788 | 85 |
| 16 | Perfection | 9.0 | 13.4 | 7.0 | 0.57 | 850 | 85 |
| 17 | Princeton P-105 | 10.5 | 12.2 | 11.9 | 0.59 | 360 | 92 |
| 18 | Rugby II | 9.5 | 16.5 | 7.3 | 0.59 | 868 | 78 |
| 19 | Total Eclipse | 9.9 | 14.3 | 6.5 | 0.53 | 790 | 75 |
| 20 | Tsunami | 8.6 | 14.3 | 6.2 | 0.54 | 573 | 92 |
| | LSD (5%) | 1.5 | 4.5 | 2.9 | 0.09 | 151 | 14 |

^{†}PL—Panicle Length (cm),
FLH—Flag Leaf Height (cm),
FLL—Flag Leaf Length (cm),
FLW—Flag Leaf Width (cm),
SY—Seed Yield (lbs acre⁻¹) and
PG—Percent Germination (%)

### Drought Tolerance

‘NE-KYB-05-001’ was tested and compared with 22 other Kentucky bluegrass cultivars in a turfgrass evaluation experiment initiated on September 05 at University of Nebraska John Seaton Anderson Turfgrass Research Facility. Irrigation was performed at a 60% ET deficit level to evaluate cultivar performance under drought stress conditions. Normal irrigation for Kentucky bluegrasses in this region is generally conducted at ETp around 80-90%.

The 2006 growing season was the establishment year for this study. ‘NE-KYB-05-001’ had the second lowest density rating during the establishment year (Table 4). This was a result of the other cultivars being seeded and ‘NE-KYB-05-001’ being established from vegetative sprigs. Seeding gave the other cultivars an advantage during the establishment year. Despite low density ratings during establishment year, ‘NE-KYB-05-001’ had the highest quality ratings in September and November, and the second highest quality rating scores in July and August. In addition to that, ‘NE-KYB-05-001’ had the darkest green genetic color rating for all of the cultivars evaluated (Table 4). With its natural and unique dark green color ‘NE-KYB-05-001’ will require less Nitrogen to maintain the same level of green color compared to other bluegrasses.

In 2007, ‘NE-KYB-05-001’ had spring density ratings that exceeded many varieties (Table 5). Its summer density ratings were ranked third along with Perfection and Caliber. These results support ‘NE-KYB-05-001’ as a cultivar that is capable of producing a dense high quality turf. ‘NE-KYB-05-001’ consistently ranked among the highest in turfgrass quality ratings during the 2007 growing season (Table 5). This trend continued in the 2008 growing season where ‘NE-KYB-05-001’ had the highest ratings for spring green up, for spring and summer density; and for quality for May and June (Table 6). ‘NE-KYB-05-001’ forms a high quality, dense, dark green turf even under deficit irrigation conditions (60% ET), demonstrating to have a high level of drought tolerance.

TABLE 4
Mean turfgrass color, density, and quality ratings for Kentucky
bluegrass cultivar evaluation study. Data are from 2006.

| | Color | | Quality | | | |
|---|---|---|---|---|---|---|
| Cultivars | August | Density August | July | August | September | November |
| Absolute | 6.7 | 9.0 | 6.7 | 5.7 | 6.3 | 5.0 |
| ‘NE-KYB-05-001’ | 9.0 | 7.3 | 8.3 | 7.7 | 8.3 | 7.0 |
| Blue Chip | 7.0 | 8.7 | 6.7 | 6.0 | 5.0 | 4.3 |
| Blue Moon | 7.3 | 9.0 | 7.3 | 6.7 | 6.7 | 5.3 |
| Caliber | 5.7 | 9.0 | 5.0 | 4.7 | 5.3 | 4.0 |
| Common | 6.0 | 8.7 | 5.7 | 5.3 | 6.0 | 4.7 |
| Everest | 9.0 | 8.7 | 8.3 | 8.0 | 8.0 | 6.7 |
| Everglade | 8.3 | 9.0 | 8.0 | 7.0 | 6.0 | 6.0 |
| Impact | 8.7 | 9.0 | 8.0 | 7.7 | 8.0 | 5.3 |
| Kenblue | 5.3 | 6.0 | 4.3 | 6.7 | 3.7 | 4.3 |
| Liberator | 8.7 | 8.0 | 8.3 | 8.0 | 8.0 | 6.3 |
| Limousine | 6.7 | 9.0 | 6.0 | 5.0 | 4.7 | 3.0 |
| Midnight | 4.0 | 9.0 | 4.3 | 3.7 | 3.7 | 4.3 |
| NuBlue | 6.7 | 9.0 | 6.7 | 5.3 | 6.0 | 5.7 |
| NuDensity | 8.7 | 9.0 | 8.7 | 7.7 | 8.3 | 6.7 |
| NuGlade | 8.3 | 9.0 | 7.7 | 6.7 | 7.7 | 6.3 |
| Park | 5.3 | 9.0 | 5.0 | 4.7 | 4.7 | 4.0 |
| Perfection | 8.3 | 9.0 | 8.0 | 7.0 | 7.7 | 6.3 |
| Princeton P-105 | 8.0 | 9.0 | 7.7 | 7.3 | 6.7 | 6.3 |
| Rambo | 8.7 | 9.0 | 8.0 | 7.7 | 6.7 | 6.0 |
| Rugby II | 7.3 | 9.0 | 6.7 | 5.3 | 6.0 | 5.0 |
| Total Eclipse | 7.3 | 9.0 | 7.3 | 6.3 | 7.3 | 5.7 |
| Tsunami | 7.7 | 9.0 | 8.0 | 8.0 | 7.3 | 6.7 |
| LSD (5%) | 0.6 | 0.5 | 0.7 | 0.6 | 0.8 | 0.5 |

TABLE 5
Mean turfgrass density, and quality ratings for Kentucky bluegrass
cultivar evaluation. Data are from 2007.

| | Density | | | Quality | | | | | |
|---|---|---|---|---|---|---|---|---|---|
| Cultivar | Spring | Summer | Fall | April | May | July | Aug | Sept | Oct |
| Absolute | 8.0 | 8.3 | 9.0 | 3.0 | 5.7 | 5.7 | 6.7 | 5.7 | 6.7 |
| ‘NE-KYB-05-001’ | 7.7 | 8.0 | 9.0 | 4.0 | 7.7 | 7.3 | 7.3 | 7.7 | 7.0 |
| BlueChip | 8.0 | 6.0 | 9.0 | 2.7 | 6.3 | 5.7 | 5.7 | 5.7 | 6.7 |
| BlueMoon | 7.0 | 6.3 | 9.0 | 2.3 | 5.7 | 6.3 | 6.3 | 5.0 | 7.3 |
| Caliber | 7.3 | 8.0 | 8.7 | 2.7 | 5.7 | 6.0 | 6.0 | 5.7 | 7.0 |
| Common | 6.7 | 6.7 | 8.7 | 2.0 | 4.0 | 5.0 | 5.0 | 6.0 | 5.3 |
| Everest | 7.3 | 6.7 | 8.7 | 2.7 | 6.7 | 6.7 | 7.3 | 7.0 | 7.0 |
| Everglade | 7.0 | 7.7 | 9.0 | 2.3 | 6.3 | 7.0 | 7.3 | 7.3 | 6.7 |
| Impact | 7.7 | 7.3 | 9.0 | 2.7 | 6.0 | 7.0 | 6.7 | 6.3 | 7.0 |
| Kenblue | 6.3 | 5.0 | 8.7 | 2.0 | 4.0 | 4.7 | 4.3 | 4.3 | 5.0 |
| Liberator | 8.0 | 8.7 | 9.0 | 4.3 | 7.0 | 7.7 | 7.7 | 6.7 | 7.3 |
| Limousine | 8.7 | 6.7 | 8.7 | 4.0 | 6.3 | 6.3 | 6.7 | 6.7 | 7.3 |
| Midnight | 6.0 | 5.3 | 9.0 | 2.0 | 3.7 | 4.3 | 4.0 | 4.0 | 4.7 |
| NuBlue | 8.0 | 7.7 | 8.7 | 4.3 | 6.0 | 6.0 | 6.0 | 6.0 | 7.3 |
| NuDensity | 7.3 | 7.3 | 8.7 | 4.0 | 6.0 | 6.7 | 7.0 | 7.0 | 6.3 |
| NuGlade | 7.0 | 8.3 | 9.0 | 2.7 | 6.0 | 6.7 | 7.3 | 6.7 | 7.7 |
| Park | 6.3 | 6.3 | 9.0 | 1.0 | 3.7 | 4.7 | 4.3 | 4.7 | 5.3 |
| Perfection | 8.0 | 8.0 | 8.7 | 2.7 | 7.0 | 7.3 | 7.7 | 6.7 | 7.3 |
| Princeton P-105 | 7.3 | 6.3 | 8.7 | 2.7 | 5.0 | 5.3 | 4.7 | 5.0 | 6.7 |
| Rambo | 7.3 | 7.0 | 8.7 | 3.0 | 6.3 | 7.3 | 7.3 | 6.0 | 7.0 |
| Rugby II | 7.7 | 7.7 | 9.0 | 2.3 | 5.7 | 5.7 | 5.7 | 5.7 | 6.0 |
| Total Eclipse | 7.3 | 6.7 | 9.0 | 3.3 | 6.7 | 7.0 | 6.7 | 6.3 | 7.3 |
| Tsunami | 7.0 | 8.7 | 9.0 | 3.0 | 6.3 | 7.3 | 8.0 | 7.0 | 7.7 |
| LSD (5%) | 0.9 | 0.5 | ns | 0.6 | 0.6 | 0.6 | 0.7 | 0.9 | 0.6 |

TABLE 6
Mean turfgrass spring green-up, density, and quality ratings for
Kentucky bluegrass cultivar evaluation. Data are from 2008.

| | Spring | Density | | Quality | | |
|---|---|---|---|---|---|---|
| Cultivar | Green-up | Spring | Summer | May | June | July |
| Absolute | 7.0 | 8.0 | 7.0 | 6.3 | 6.7 | 6.3 |
| ‘NE-KYB-05-001’ | 8.3 | 8.7 | 8.3 | 7.7 | 7.3 | 6.0 |
| Blue Chip | 7.0 | 8.0 | 7.0 | 5.7 | 4.7 | 5.3 |
| Blue Moon | 6.3 | 8.0 | 6.3 | 6.7 | 6.7 | 6.7 |
| Caliber | 7.3 | 7.3 | 7.3 | 4.7 | 3.0 | 5.0 |
| Common | 6.3 | 7.7 | 6.3 | 4.3 | 2 7 | 4.0 |
| Everest | 6.7 | 8.3 | 6.7 | 6.7 | 7.0 | 6.7 |
| Everglade | 6.7 | 8.0 | 6.7 | 7.0 | 5.7 | 6.0 |
| Impact | 7.0 | 8.3 | 7.0 | 6.7 | 7.0 | 6.7 |
| Kenblue | 5.0 | 6.0 | 5.0 | 4.0 | 2.7 | 4.0 |
| Liberator | 7.7 | 8.3 | 7.7 | 6.7 | 6.3 | 7.0 |
| Limousine | 7.7 | 8.7 | 7.7 | 5.7 | 5.3 | 4.3 |
| Midnight | 5.7 | 6.0 | 5.7 | 3.7 | 2.3 | 4.0 |
| NuBlue | 7.3 | 7.7 | 7.3 | 5.3 | 3.7 | 5.3 |
| Nuoensity | 7.3 | 8.7 | 7.3 | 7.0 | 7.0 | 7.3 |
| NuGlade | 6.3 | 8.0 | 6.3 | 6.7 | 6.0 | 5.7 |
| Park | 7.3 | 7.7 | 7.3 | 4.0 | 2.0 | 4.0 |
| Perfection | 6.3 | 8.3 | 6.3 | 7.0 | 6.7 | 6.7 |
| Princeton P-105 | 7.0 | 8.0 | 7.0 | 6.7 | 6.0 | 5.7 |
| Rambo | 7.0 | 8.3 | 7.0 | 7.3 | 7.7 | 7.0 |
| Rugby II | 6.7 | 7.7 | 6.7 | 6.7 | 6.0 | 5.7 |
| Total Eclipse | 7.0 | 7.7 | 7.0 | 6.3 | 6.0 | 6.0 |
| Tsunami | 6.7 | 8.0 | 6.7 | 6.7 | 6.7 | 7.0 |
| LSD 5% | 0.8 | 0.5 | 0.7 | 0.6 | 0.5 | 0.6 |

### Shade Tolerance

An experiment was initiated in 2006 at the University of Nebraska John Seaton Anderson Turfgrass Research Facility located near Mead, Nebr. with ‘NE-KYB-05-001’, Thermal Blue, and Midnight to compare shade tolerance among these three bluegrasses. Plots were mowed weekly at 2.5 inches and clippings were removed. Plots received 3.0 lbs N per 1000 ft² per growing season with applications made at 1.0 lbs per 1000 ft² in May, September, and October. No fungicides or insecticides were applied to the plots. Post emergence herbicides were applied as needed to prevent weed encroachment problems. All 3 varieties were maintained at 60% shade conditions, during the entire experiment. Shade conditions were achieved by using a special shade cloth which provided 60% reduction of incident sunlight.

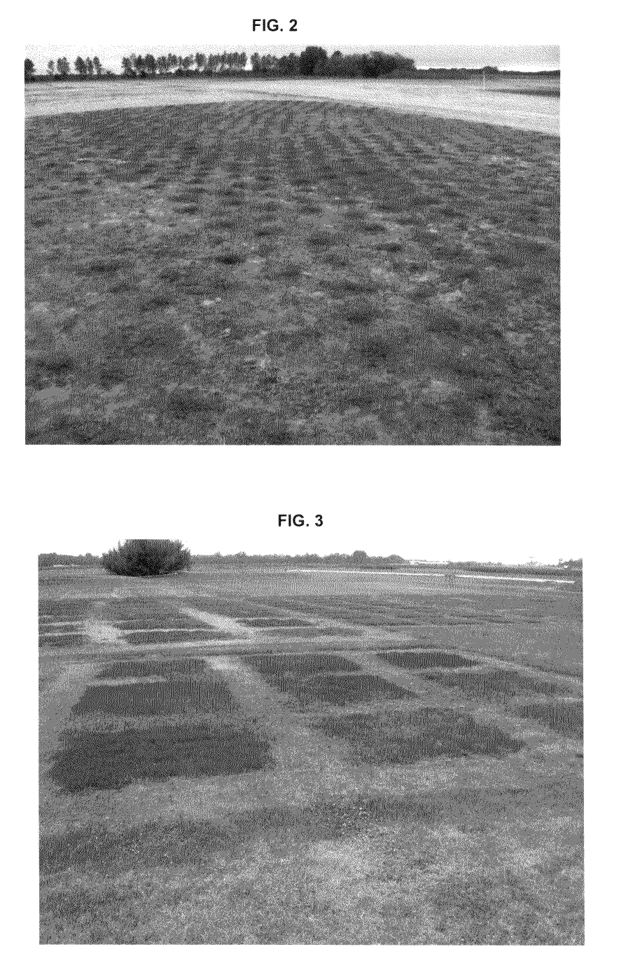

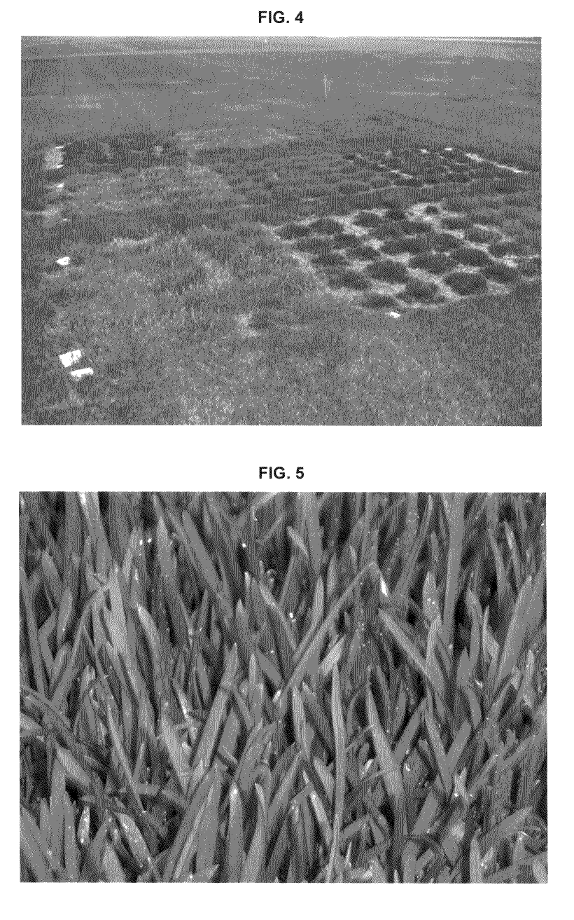

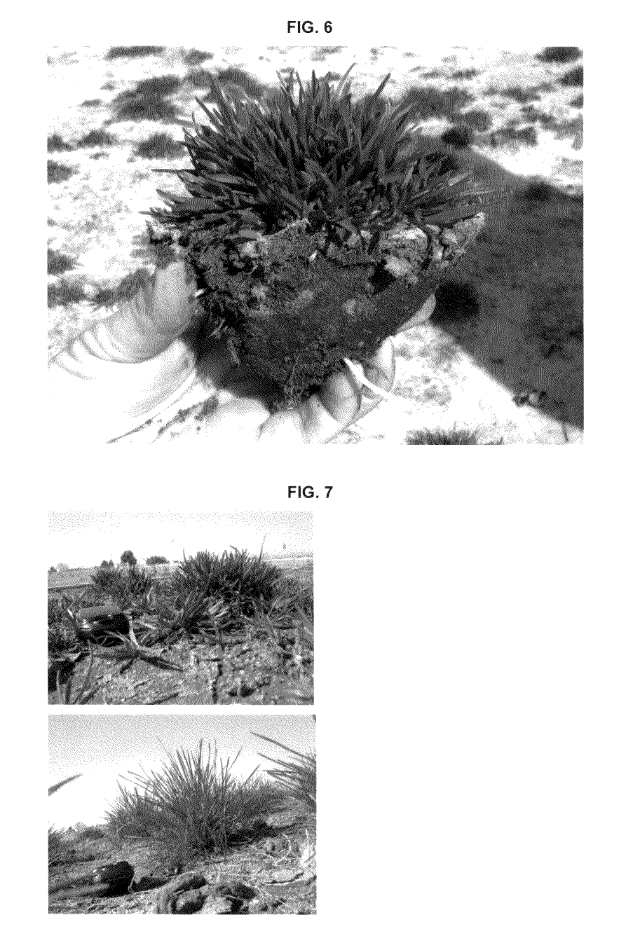

The Analysis of Variance (ANOVA) showed grasses differed significantly at 1% level in spring green-up, color, density and quality when exposed to shade conditions (Table 7). ‘NE-KYB-05-001’ maintained a darker green color and greened up earlier than Thermal Blue or Midnight (Table 8). ‘NE-KYB-05-001’ had superior turfgrass quality and density under the shaded conditions of this study. The higher turfgrass quality was primarily a result of ‘NE-KYB-05-001’ maintaining darker green color, better density and lower heights than Thermal Blue or Midnight (FIG. 2). Reduced green color and etiolated growth are common responses of plants lacking tolerance to reduced light intensity associated with shaded growing conditions. ‘NE-KYB-05-001’ demonstrated plant growth responses associated with superior shade tolerance.

TABLE 7
Analysis of variance for three Kentucky bluegrass cultivars grown at
60% shade at University of Nebraska Research Facility near Mead, NE.
Data are from 2008.

| | | Mean Squares | | | | | | |
|---|---|---|---|---|---|---|---|---|
| | | Spring | | | | | | |
| | | Green-up | Color | Density | | Quality | | |
| Source | df | March | June | Spring | Summer | May | June | July |
| Rep | 2 | 2.33 | 0.78 | 0.11 | 0.11 | 0.11 | 0.33 | 0.44 |
| Genotypes | 2 | 4.3** | 23.11** | 5.44** | 9.78** | 11.44** | 17.33** | 14.78** |
| Error | 4 | 0.17 | 0.11 | 0.11 | 0.11 | 0.11 | 0.17 | 0.28 |
| CV (%) | | 5.6 | 8.7 | 4.6 | 5.2 | 5.8 | 7.7 | 10.1 |

^{**}Significant at the 0.01 level

TABLE 8
Mean turfgrass spring green-up, color, density and quality ratings for
three Kentucky bluegrass cultivars grown in a shade evaluation study
located at the University of Nebraska John Seaton Anderson Turfgrass
Research Facility near Mead, NE. Plots were maintained under a shade
environment of 60% reduction of incident sunlight. Data are from 2008.

| | Mean | | | | | | |
|---|---|---|---|---|---|---|---|
| | Spring Green-up | Color | Density | | Quality | | |
| Genotypes | March | June | Spring | Summer | May | June | July |
| “NE-KYB-05-001” | 8.7 | 8.7 | 8.7 | 8.3 | 8.0 | 8.0 | 7.7 |
| Midnight | 6.3 | 3.3 | 6.0 | 5.0 | 4.3 | 3.3 | 3.3 |
| Thermal Blue | 7.0 | 4.7 | 7.0 | 6.0 | 5.0 | 4.7 | 4.7 |

That which is claimed is:

1. ‘NE-KYB-05-001’, is a new and distinct variety of Kentucky bluegrass, substantially as herein illustrated and described, characterized by its distinctive and unique combination of several characteristics such as: exclusively vegetative propagation, dark green foliage, shorter leaf length, improved shade and drought tolerance and slow vertical growth habit.
